(19) United States
(12) Patent Application Publication
(10) Pub. No.: US 2002/0119107 A1
(43) Pub. Date: Aug. 29, 2002

(54) Method for protecting and restoring skin using selective MMP inhibitors

(76) Inventors: James Varani, Ann Arbor, MI (US); Gary J. Fisher, Ann Arbor, MI (US); John J. Voorhees, Ann Arbor, MI (US)

Correspondence Address: Hopgood, Calimafde, Judlowe & Mondolino, 60 East 42nd Street, New York, NY, 10165, US

(21) Appl. No.: 09/740,242

(22) Filed: Dec. 18, 2000

(51) Int. Cl. A61K 7/42; A61K 7/00

(52) U.S. Cl. 424/59; 424/400; 424/401

(57) ABSTRACT

The invention is based on selective inhibition of the enzyme (MMP-1), which causes the dermal matrix damage in humans, while sparing the enzyme(s) (MMP-9 and perhaps MMP-2) which not only do not cause the damage (based on extrapolation from our in vitro collagen gel system to real skin) but actually “clear away” the damage produced by MMP-1 to restore normal function to the skin. Matrix metalloproteinase-1 (MMP-1; fibroblast collagenase) is induced by UV radiation from the sun and is naturally elevated in old age. Human fibroblasts exposed to the degradation products of MMP-1 contract collagen, but when this debris is removed from their environment, the fibroblasts behave normally. Inhibiting MMP-1 but sparing enzymes that remove the debris improves human skin after onslaught from solar UV radiation, old age, and acne.

### BACKGROUND OF THE INVENTION

1. Field of the Invention

This invention relates to the use of compositions administered to human skin for its protection from the effects of aging and ultraviolet light and to restore the skin from exposure to such effects.

2. The State of the Art.

Our prior patents for photoaging, U.S. Pat. Nos. 5,837,224 and 6,130,254, and for chronological aging U.S.______ (Ser. No. 09/028,435, filed Feb. 24, 1998), the disclosures of which are incorporated herein by reference, describe the effects of ultraviolet radiation (UV) and of time (age) on human skin. Whether subject to UV radiation or the effects of time, matrix metalloproteinases (MMPs) are induced in human skin. These enzymes degrade collagen in the dermal matrix, which is slowly repaired. Because humans are often exposed to UV radiation, and are constantly aging, this degradation and repair process is constantly repeating. It is believed by us that imperfect repair leads to microdefects or microscars in the dermal matrix, and these eventually accumulate to the point where the skin has the clinical effects of photoaging and/or chronoaging. It should be evident that on certain parts of the body, such as the face, hands, and forearms, the skin is almost always subjected to a combination of photoaging and chronological aging, while on other parts of the body the predominant effect is chronological aging.

In photodamage and in natural aging (chronoaging), the collagenous matrix of the dermis is degraded. Dermal fibroblasts interact with this damaged material. These changes can be appreciated at the histological (light-microscopic) level and at the ultrastructural (electron microscopic) level, as well as biochemically. These changes are thought to underlie the clinical deficits seen in naturally-aged skin and in photodamaged skin. Damage to the collagenous matrix of the dermis has been observed at both the light and electron microscopic levels in photoaged skin. Reductions in both the number and size of the collagen fiber bundles as well as ultrastructural abnormalities in the collagen fibrils themselves have been noted. However, the presence of elastotic material often “masks” structural evidence of damage, and makes quantification of damage difficult. Consistent with past reports large bundles of collagenous fibers were present throughout the dermis of sun-protected skin, such as from the hip. Healthy fibroblasts in intimate contact with the collagen bundles could be seen under both light microscope, in FIG. 1A, and electron microscope, in FIG. 1C. In contrast, severely photodamaged skin, such as on the forearm, was characterized by the presence of fewer bundles of collagen, and many individual, disorganized fibers. The space between the collagen bundles, where not occupied with elastotic material, was filled with mostly-acellular debris. Instead of being in contact with intact collagen, many of the fibroblasts in the damaged skin were surrounded by the debris. Some of the cells demonstrated a rounded rather than elongated morphology and, in some cases, there were aggregates of two or more cells. These features are seen in light microscopy, in FIG. 1B, and with electron microscopy, in FIG. 1D. Thus, electron microscopy proved useful for identifying a reduction in the relative amount of intact collagen in the photodamaged skin, the presence of acellular debris, and contact/interaction of dermal fibroblasts with this debris rather than with intact collagen.

Ultrastructural analysis also provided evidence of damage to the collagen fibers themselves. While some of the collagen fibers in photodamaged skin demonstrated the same overall width (approximately 1500 A) and periodicity as in sun-protected skin, others appeared shortened and thinned. To quantitatively assess collagen fragmentation, we took advantage of the fact that intact collagen is insensitive to in vitro hydrolysis by α-chymotrypsin, while collagen which has been partially degraded in vivo is susceptible to further hydrolysis by this enzyme in vitro. Digestion of partially degraded collagen by α-chymotrypsin liberates collagen fragments from the tissue, and the liberated collagen fragments can be quantified by hydroxyproline measurement. Hydroxyproline content after α-chymotrypsin digestion is, therefore, a measure of partially-degraded collagen in the tissue. FIG. 2 compares amounts of hydroxyproline released by α-chymotrypsin treatment of matched samples of severely photodamaged forearm skin and sun-protected hip skin from nine individuals. The amount released from photodamaged skin was 3.6-fold higher than the amount released from matched sun-protected skin.

M. Whittaker et al., “Design and Therapeutic Application of Matrix Metalloproteinase Inhibitors”, Chem. Rev. 1999, 99, 2735-2776, the disclosure of which describes the various classes of MMP inhibitors, design philosophy (structure-based versus substrate-based), and provide examples of MMP inhibitors and their ability to inhibit specific MMPs. There are twenty three MMPs, about six of which are most important from the point of view of human skin with respect to photoaging and chronoaging. These MMPs and their principal substrates (from Whittaker et al.) are:

| Enzyme | Principal Substrate |
|---|---|
| MMP-1 (fibroblast collagenase) | collagen Types I, II, III, VI, and X |
| MMP-2 (galatinase A; 72 kDa | collagen Types IV, V, VII, X, elastin |
| gelatinase) | |
| MMP-3 (Stromelysin-1) | proteoglycan, collagen Types III, IV, V |
| | and IX, gelatins, pro-MMP-1 |
| MMP-8 (neutrophil collagenase) | collagen Types I, II, and III |
| MMP-9 (gelatinase B; 92 kDa | collagen Types IV and V, gelatins |
| gelatinase) | |
| MMP-12 (metalloelastase) | elastin |
| MMP-13 (collagenase-3) | collagen Types I and III, gelatin |

One could also argue that MMP-14 and MMP-16, both of which degrade pro-MMP-2, are also important.

MMP-1 cleaves collagen Type I, the main component of the dermal matrix into ¼ and ¾ fragments. Both of these fragments are further cleaved to small pieces by MMP-9 and MMP-2. Neither MMP-9 or MMP-2 cleaves intact collagen.

### SUMMARY AND OBJECTS OF THE INVENTION

What we show here for the first time is that when fibroblasts are maintained on collagen that has been cleaved by MMP-1, their behavior is affected. Specifically, type I procollagen synthesis is reduced. However, when fibroblasts are maintained on collagen that has been cleaved by a combination of MMP-1 and MMP-9, the detrimental effect on their behavior seen previously when they were maintained in the presence of only MMP-1 cleavage products is mitigated. Specifically, type I procollagen production of fibroblasts is not inhibited when the fibroblasts are exposed to the degradation products of MMP-1 on collagen if MMP-9 is also present.

Thus, this invention is based on selective inhibition of the enzyme (MMP-1) which causes the matrix damage while sparing the enzyme(s) (MMP-9 and perhaps MMP-2) which not only do not cause the damage (based on extrapolation from our in vitro collagen gel system to real skin) but actually “clear away” the damage produced by MMP-1 to restore normal function to the skin.

While not desirous of being constrained to a particular theory, we hypothesize that the fragments generated from intact collagen by MMP-1 exert the inhibitory influence and that the positive effects of MMP-9 reflect further degradation of the fragments generated by the action of MMP-1.

Accordingly, the main object of this invention is provide selective inhibition of MMP-1, induced especially by exposure of human skin to UV radiation and by the chronological aging process, while allowing MMP-9 and/or MMP-2 to degrade the collagen fragments resulting from cleavage by MMP-1.

Another object of this invention is to provide a composition comprising a combination of a UVA blocker, a UVB blocker, and an inhibitor selective for MMP-1.

Yet another object of this invention is to provide an improved method for practicing the aforementioned patents and applications relating to photoaging and chronoaging, which comprises topically applying to human skin an inhibitor selective for MMP-1.

### BRIEF DESCRIPTION OF THE FIGURES

FIG. 1 depicts light microscopy (FIGS. 1A and 1B) and electron microscopy (FIGS. 1C and 1D) of in vivo skin biopsies from sun-protected (“hip”) skin and sun exposed (“forearm”) skin.

FIG. 2 compares amounts of hydroxyproline released by α-chymotrypsin treatment of matched samples of severely photodamaged forearm skin and sun-protected hip skin.

FIG. 3 shows type I procollagen protein expression in severely photodamaged skin (“forearm”) versus sun-protected skin (“hip”).

FIG. 4 shows the number of cells expressing type I procollagen (α1) MRNA in severely photodamaged forearm skin when compared with sun-protected skin from the hip.

FIGS. 5A and 5B depict the results as to the number of cells (5A) and type I procollagen (5B) existing after fibroblasts were extracted from sun-damaged (“forearm”) skin and from sun-protected (“hip”) and cultured in vitro.

FIG. 6 depicts results of determining the ability of the bacterial collagenase and human skin collagenase to degrade monomeric collagen.

FIG. 7A is a light microscopy of a cross-section through a plated, untreated gel, where fibroblasts can be seen on the upper surface and others are relatively uniformly dispered in the gel; FIG. 7B is a similar a cross section on degraded collagen where cell-cell aggregation can be seen; FIG. 7C is an electron microscopy view of fibroblasts with their numerous processes on untreated collagen gel; FIG. 7D is a similar electron microscopy view of fibroblasts surrounded by the collagen degradation debris showed decreased process formation, and few contacts between the cells and intact collagen fibers.

FIG. 8A shows the dose-dependent relationship of the collagenases to the amount of collagen contraction achieved FIG. 8B presents evidence that the metalloproteinases applied to the collagen gels were the cause of the collagen contraction; and FIG. 8C shows that collagen contraction was dependent on fibroblast activity.

FIG. 9A shows cell growth and FIG. 9B shows the amount of type I procollagen produced on partially degraded versus intact collagen, regardless of whether the collagen was degraded by the bacterial collagenase or the human skin collagenase.

Lane 1 of FIG. 10A is intact collagen with the α1(I) and α2(I) bands for collagen visible; Lane 2 of FIG. 10A shows the ¾ and ¼ fragments after digestion of collagen by MMP-1; and Lanes 3 and 4 of FIG. 10A show that MMP-2 and MMP-9 did not degrade the collagen as did MMP-1.

Lane 1 of FIG. 10B shows the resulting gelatin made by heating collagen to 60° C. for five minutes; Lanes 2 and 3 of FIG. 10B show the degradation products when gelatin is exposed, respectively, to MMP-2 and to MMP-9.

Lane 1 of FIG. 10C is the control collagen; Lane 2 shows the degradation products when MMP-1 is presented to collagen; Lane 3 shows the degradation products of collagen when both MMP-1 and MMP-2 are present; and Lane 4 of FIG. 10C shows the degradation products of collagen when both MMP-1 and MMP-9 are present; namely, the ¾ and ¼ fragments produced by MMP-1 disappear.

FIGS. 11A through 11C show, respectively, the ability of the fibroblasts to contract the collagen (11A), the proliferation of the fibroblasts (11B), and their production of procollagen (11C).

### DETAILED DESCRIPTION OF SPECIFIC EMBODIMENTS

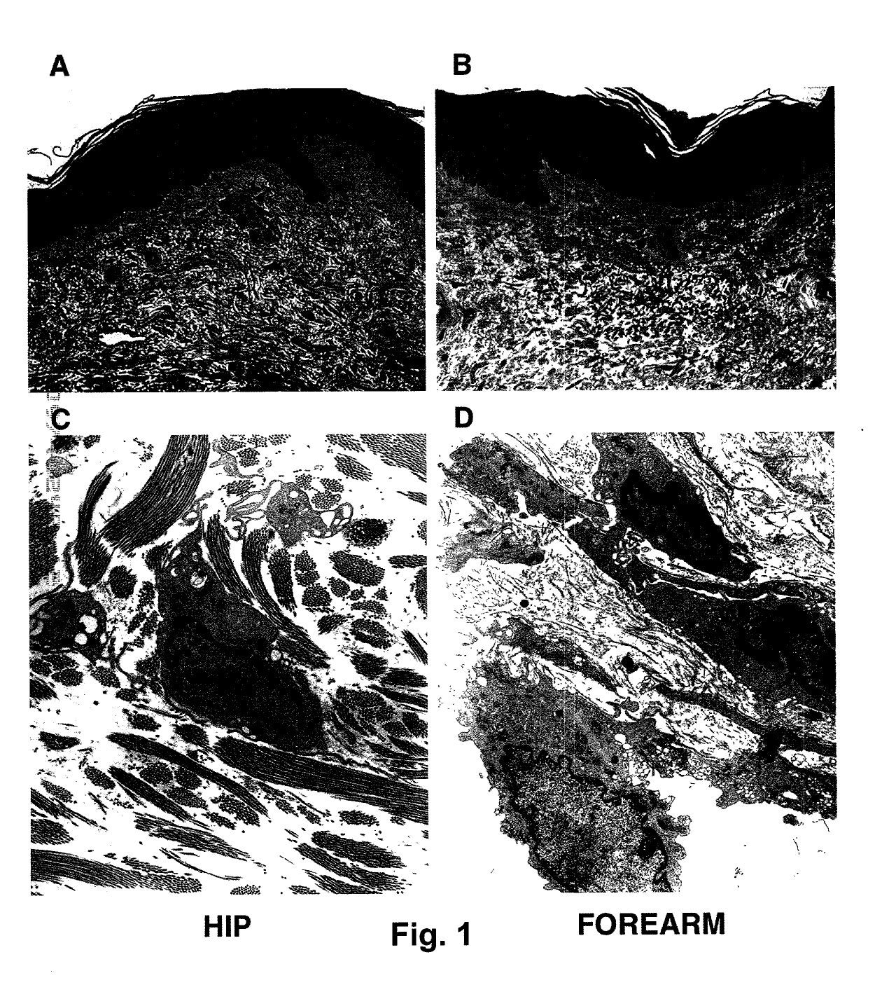

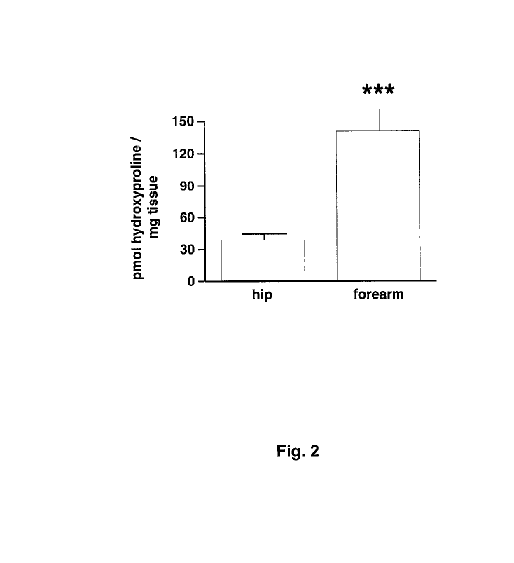

In both photodamaged and naturally-aged skin, the collagenous matrix of the dermis is degraded. The changes shown in FIGS. 1A-1D and FIG. 2 are believed to underlie the clinical deficits seen in naturally-aged and photodamaged skin. For example, this damage to the collagenous matrix is thought to underlie the coarse, rough, wrinkled appearance of photoaged skin. How collagen damage is brought about during photoaging is not fully understood. Exposure of skin to UV irradiation transiently up-regulates production of MMPs that degrade skin collagen, as observed by Fisher GJ et al., “The molecular basis of sun-induced premature skin ageing and retinoid antagonism,” Nature (London) 1966: 379:335-338; Fisher GJ et al., “Pathophysiology of premature skin aging induced by ultraviolet light,” New Eng. J. Med. 1977: 337:1419-1428. Repeated MMP induction over years or decades likely gives rise to the damage seen in the matrix of chronically sun-exposed skin.

Damage to the collagenous matrix of the dermis has been observed at both the light and electron microscopic levels in photoaged skin, by us and others, since at least as early as 1966. Reductions in both the number and size of the collagen fiber bundles, as well as ultrastructural abnormalities in the collagen fibrils themselves, have been noted. However, the presence of elastotic material often “masks” structural evidence of damage, and makes quantification of damage difficult. Transmission electron microscopy (TEM) was used to compare structural features of the collagen in severely photodamaged skin and in matched sun-protected skin from the same individuals. Consistent with these past reports, large bundles of collagenous fibers were present throughout the dermis of sun-protected skin. Healthy fibroblasts in intimate contact with the collagen bundles could be seen in FIGS. 1A (light micrograph) and 1C (TEM). In contrast, severely photodamaged skin was characterized by the presence of fewer bundles of collagen, and many individual, disorganized fibers. The space between the collagen bundles, where not occupied with elastotic material, was filled with mostly-acellular debris. Instead of being in contact with intact collagen, many of the fibroblasts in the damaged skin were surrounded by the debris. Some of the cells demonstrated a rounded rather than elongated morphology and, in some cases, there were aggregates of two or more cells. These features of sun-exposed skin are shown in FIGS. 1B (light micrograph) and 1D (TEM). Thus, photodamaged skin evidences a reduction in the relative amount of intact collagen, the presence of acellular debris, and contact/interaction of dermal fibroblasts with this debris rather than with intact collagen.

Ultrastructural analysis also provided evidence of damage to the collagen fibers themselves. While some of the collagen fibers in photodamaged skin demonstrated the same overall width (approximately 1500 Å) and periodicity as in sun-protected skin, others appeared shortened and thinned. To quantitatively assess collagen fragmentation, we took advantage of the fact that intact collagen is insensitive to in vitro hydrolysis by α-chymotrypsin, while collagen which has been partially degraded in vivo is susceptible to further hydrolysis by this enzyme in vitro. Digestion of partially, in vivo degraded collagen by α-chymotrypsin liberates collagen fragments from the tissue, and the liberated collagen fragments can be quantified by hydroxyproline measurement. Therefore, hydroxyproline content after α-chymotrypsin digestion of these samples is a measure of partially-degraded collagen in the tissue. FIG. 2 compares amounts of hydroxyproline released by α-chymotrypsin treatment of matched samples of severely photodamaged forearm skin and sun-protected hip skin from nine individuals, showing that the amount released from photodamaged skin was 3.6 times greater than the amount released from matched sun-protected skin.

Although proteolytic attack on structural collagen is clearly part of the overall process, failure to replace damaged collagen with newly-synthesized collagen also contributes to the progressive degenerative changes that occur in the connective tissue of sun-exposed skin over time. NEJM 1977:337 op. cit.; Griffiths CEM et al., “Restoration of collagen formation in photodamaged human skin by tretinoin (retinoic acid),” New Eng. J. Med. 1993: 329: 530-534; Talwar HS et al., “Reduced type I and type III procollagens in photodamaged adult human skin,” J. Invest. Dermatol. 1995: 105:285-290. We have shown there is a concommittant decrease in collagen biosynthesis in human skin after exposure to UVA and/or UVB radiation. U.S. Prov. Pat. Appln. No. 60/080437, filed Apr. 2, 1998, and co-pending U.S. patent application Ser. No. 09/285860, filed Apr. 2, 1999, the disclosures of which are incorporated herein by reference.

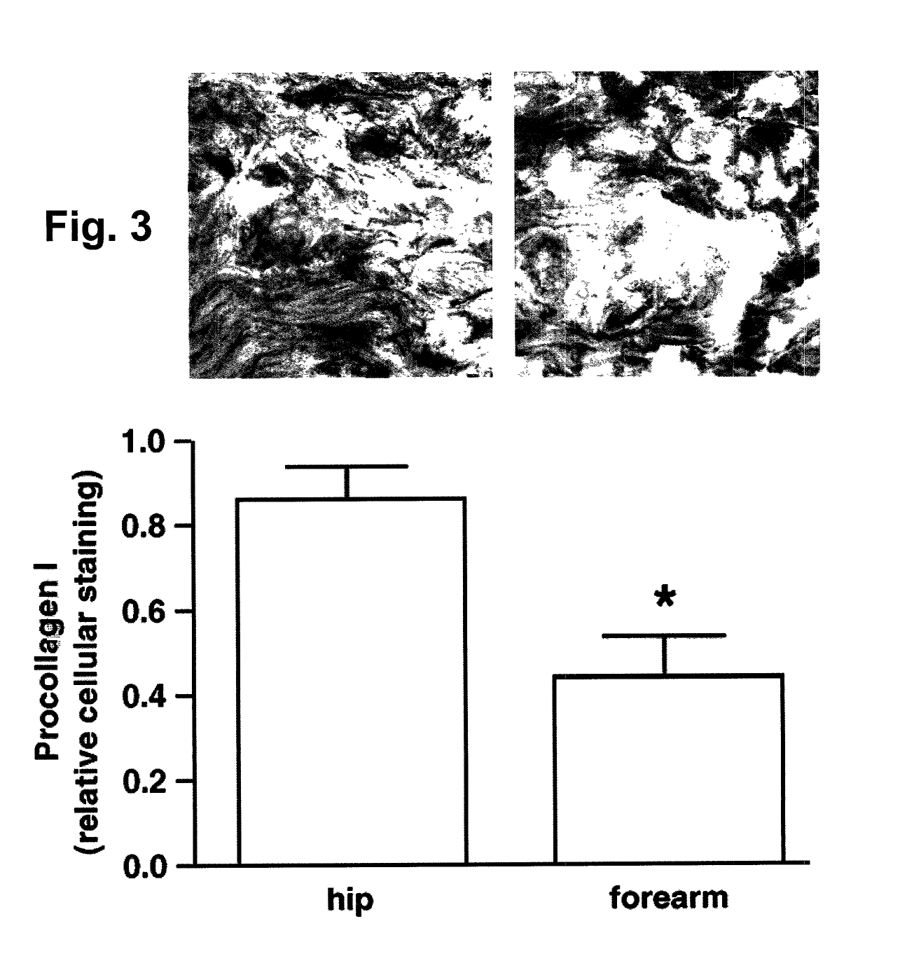

Mechanisms underlying this decreased collagen synthesis by fibroblasts in severely photodamaged skin are not completely understood in the present state of the art. Based on results presented herein, we conclude that while fibroblast synthesis of type I procollagen is greatly diminished in photoaged human skin in vivo, the growth capacity and synthesis of type I procollagen by fibroblasts from sun-damaged skin and age-matched sun-protected skin are indistinguishable when the cells are removed from the skin and examined in vitro. Since our data indicate that equivalent numbers of fibroblasts can be isolated from photodamaged skin and sun-protected skin, and since our data are based on results of multiple isolates from both tissue sites (from 9 different individuals), it is unlikely that the in vitro data are skewed by a small sub-population of cells in the photodamaged skin which grow out from the tissue and demonstrate the same phenotype as fibroblasts from sun-protected skin in vitro. Rather, these studies indicate that reduced procollagen production observed in vivo in severely sun-damaged skin is not due to reduced synthetic capacity of the fibroblasts per se. Consistent with these observations, it has been demonstrated previously that synthesis of collagen (as well as fibronectin) is low or undetectable in organ cultures of sun-exposed skin relative to organ cultures of healthy young skin. Varani J et al., “All-trans retinoic acid (RA) stimulates events in organ-cultured human skin that underlie repair,” J. Clin. Invest. 1994: 94:1747-1753; Varani J et al., “Molecular mechanisms of intrinsic skin aging and retinoid-induced repair and reversal,” J. Invest. Dermatol. (Suppl.) 1988: 3:57-60. Synthesis of both of these matrix components is normalized when the organ cultures are treated with concentrations of all-trans retinoic acid that induce collagen expression in photoaged skin in vivo. Kang S et al., “Application of retinol to human skin in vivo induces epidermal hyperplasia and cellular retinoid-binding proteins characteristic of retinoic acid but without measurable retinoic acid levels or irritation,” J. Invest. Dermatol. 1995: 105:549-556. Taken together with these previous observations, the present finding that fibroblasts in severely photoaged skin are not intrinsically damaged (with respect to collagen production) provides a rationale for therapeutic intervention with agents such as all-trans retinoic acid to stimulate collagen synthesis in order to repair photodamaged skin. Moreover, because the fibroblasts are not intrinsically incapable of procollagen production, there would appear to be something in the environment of photodamaged (and possibly chronologically-aged) skin that detrimentally affects the fibroblasts. FIGS. 3, 4, and 5, discussed below, support this contention.

Because dermal fibroblasts do not appear to be intrinsically damaged in severely photoaged skin, it follows that inhibitory influences within the in vivo environment of severely photodamaged skin may act in some way to prevent cells, which are inherently capable of elaborating collagen, from doing so. In vitro studies carried out with intact and partially-degraded collagen gels, described below, support this suggestion. When skin fibroblasts (either neonatal or adult) were added to polymerized collagen, they rapidly attached and spread, and they continued to proliferate and synthesize type I procollagen. In contrast, when fibroblasts were added to collagen gels that had been exposed to collagenase, cell growth and type I procollagen synthesis were reduced. While extrapolating from in vitro experiments to what may occur in vivo is difficult, these data provide evidence that fibroblast functions which are important for maintenance of dermal connective tissue are inhibited in the presence of fragmented collagen. It should be noted that while both cell growth and Type I procollagen production were reduced on the degraded collagen, the decrease in procollagen production was greater.

As noted in our patents for photoaging, U.S. Pat. Nos. 5,837,224 and 6,130,254, and for chronological aging U.S. ______ (Ser. No. 09/028,435, filed Feb. 24, 1998), MMPs are induced by exposure of human skin to UV radiation, even at UV levels below those that cause erythema (sunburn). These destructive enzymes also are present at elevated levels in old, sun-protected skin. Those patents and patent application generally teach the use of retinoids, such as retinoic acid and retinol, and direct-acting MMP inhibitors such as Batimastat, as well as other compounds having MMP inhibitory activity, for preventing the UV-induced presence of MMPs and for decreasing the naturally-elevated MMP levels in elderly skin. The results presented herein show that those teachings, while accurate, are incomplete in light of our present findings that among the enzymes present in the human skin, MMP-1 (intersitial collagenase) is sufficient to produce the collagen degradation that leads to reduced type I procollagen production, but that when MMP-9 (92-kD gelatinase B) is included along with MMP-1, the negative behavior of the cells is mitigated.

Based on an understanding of mechanisms of photoaging and natural aging in our patents and patent applications mentioned herein, several approaches have been proposed to inhibit or reverse skin aging by interfering with these mechanism. For example, retinoids, which are known to inhibit and reverse clinical features of damaged skin are postulated to work, in part, by providing a broad-spectrum inhibition of matrix metalloproteinase formation in both naturally-aged and photoaged skin. Retinoid inhibition of matrix metalloproteinase elaboration may result from preventing activation of the AP-1 transcription complex. EGF-receptor antagonism provides another way to prevent matrix metalloproteinase-induced skin damage; this also would be expected to work by providing a broad inhibition of matrix metalloproteinase up-regulation. Broad-spectrum matrix metalloproteinase inhibitors could also be expected to work by virtue of their ability to inhibit the function of these enzymes. All of these approaches are based on broadly inhibiting matrix metalloproteinase production or function. The present invention is different. It is based on selective inhibition of the enzyme (MMP-1) which causes the matrix damage while sparing the enzyme(s) (MMP-9 and perhaps MMP-2) which not only do not cause the damage (based on extrapolation from our in vitro collagen gel system to real skin) but actually “clear away” the damage produced by MMP-1 to restore normal function to the skin.

Accordingly, we suggest following the teachings of Whittaker et al. and the like to determine which MMPs a given inhibitor selectively inhibits, but not those teachings for defining the substrate for a particular MMP. For example, in the Whittaker et al. article over one hundred MMP inhibitors are described by structure, but only compound 28 (referenced as described by Miller, A. et al. in Bioorg. Med. Chem. Lett., 1997, 7, 193) and compound 53 (described in WO9817655 and in Chem. Absir., 1999, 128, 308398) are specifically disclosed as providing a sufficiently selective inhibition of MMP-1 over MMP-9. For example, compound 28 has an IC₅₀ value of 20 nM for MMP-1 and 2000 nM for MMP-9 (100:1 selectivity), and compound 53 has an IC₅₀ value of 6 nM for MMP-1 and 2000 nM for MMP-9 (333:1 selectivity). Compounds such as compound 52 (Ro 32-3555) in the Whittaker et al. article have a selectivity of about 20:1 (3 nM for MMP-1 versus 59 nM for MMP-9), which may likely be sufficient from a clinical level because, as discussed above, it is the degradation products of the MMP-1 cleavage of Type I collagen that appear to be detrimental to the health of the skin, and so as long as there is a reasonable amount of MMP-9 activity, those products will be cleared from the dermal matrix. As mentioned above, it would not be detrimental to forego inhibition of MMP-2, and compound 53 in Whittaker et al. shows an IC₅₀ value of 6 nM for MMP-1 and 900 nM for MMP-2 (150:1 selectivity). Compound 52 (Ro 32-3555) shows an IC₅₀ value of 3 nM for MMP-1, 154 nM for MMP-2, and 59 nM for MMP-9; at least a 20:1 selectivity for the collagenase over the gelatinases.

While the above experiments were performed with respect to MMP-1, MMP-8 and MMP-13 are likely to be as detrimental as MMP-1. Accordingly, compounds such as compounds 52 and 53 mentioned above, which show a selectivity of MMP-8 over MMP-9 of about 15:1 and 10:1, respectively, and compound 80 (over 350:1 selectivity), are each likely to be useful in this invention.

Our prior application No. 576,597, filed May 22, 2000 (the disclosure of which is incorporated herein by reference) describes preventing acne-induced inflammation and scarring by inhibiting MMP-8 and MMP-1. With the benefit of the present invention, an improved treatment would be the inhibition of these MMPs with a compound selective for their inhibition with respect to MMP-9 and optionally MMP-2.

Our prior provisional application No. 60/213,940, filed Jun. 26, 2000 (the disclosure of which is incorporated herein by reference) describes preventing MMP induction by the topical application of an EGF-R protein tyrosine kinase inhibitor. With the benefit of the present invention, an improved treatment would be the concommitant inhibition of these MMPs with a compound selective for their inhibition with respect to MMP-9 and optionally MMP-2.

Experimental

Skin samples from severely sun-damaged forearm skin and matched sun-protected hip skin from the same individuals were assessed for type I procollagen gene expression by in situ hybridization and for type I procollagen protein by immunostaining. Both mRNA and intracellular protein were reduced (approximately 65% and 57% respectively) in photodamaged forearm skin compared to sun-protected hip skin.

We next investigated whether reduced type I procollagen production was due to inherently reduced capacity of skin fibroblasts in severely photodamaged forearm skin to synthesize procollagen, or whether the environment within photodamaged skin acts to down-regulate type I procollagen synthesis in these fibroblasts. For these studies, fibroblasts from photodamaged skin and matched sun-protected skin were established in culture. Equivalent numbers of fibroblasts were isolated from the two skin sites. Fibroblasts from these two sites had similar growth capacities and produced virtually identical amounts of type I procollagen protein in culture.

Thus, the reduction in type I procollagen synthesis by fibroblasts could well be due to the photodamaged environment of the skin. In sun-protected skin, collagen fibrils exist as a highly-organized matrix. Fibroblasts are found within the collagenous matrix, and in close opposition with collagen fibers. In photodamaged skin, collagen fibrils are shortened, thinned and disorganized, and the amount of partially-degraded collagen is approximately 3.6-fold greater in photodamaged skin than in sun-protected skin. In addition, some fibroblasts are surrounded by debris (i.e., the degraded collagenous matrix of the dermis).

To model this environment in photodamaged skin, human skin fibroblasts were cultured in vitro on intact collagen, or on collagen that had been partially degraded by exposure to collagenolytic enzymes. Collagen that had been partially-degraded by exposure to collagenolytic enzymes (from either bacteria or from human skin) underwent contraction in the presence of dermal fibroblasts, while intact (non-degraded) collagen did not. Fibroblasts cultured on collagen that had been degraded by exposure to a collagenolytic enzyme demonstrated reduced proliferative capacity (about 20% reduction on collagen degraded by either bacterial collagenase or human skin collagenase) and synthesized less type I procollagen on a per cell basis. These findings indicate that (i) fibroblasts from photoaged and sun-protected skin are similar in their capacities for growth and for producing type I procollagen, and (ii) fibroblasts in the environment of degraded collagen have reduced type I procollagen synthesis.

A total of 42 individual volunteers (22 males and 20 females) were characterized by the presence of severe photodamage on their forearms based on clinical criteria—e.g., coarseness of the skin and degree of wrinkling. The age range was 46-83 years, with the average age being 69 years. Replicate 4-mm full-thickness punch biopsies of forearm and sun-protected hip skin were obtained from each individual. (All procedures involving human subjects were approved by the University of Michigan Institutional Review Board, and all subjects provided written informed-consent prior to their inclusion in the study.) In 18 of these individuals, we were able to obtain biopsies of sun-protected underarm skin as well as skin from the other two sites (forearm and hip). Overall, sun-protected skin from the underarm and hip areas was similar in regard to the parameters of collagen fragmentation, fibroblast isolation rates, proliferation and collagen synthesis.

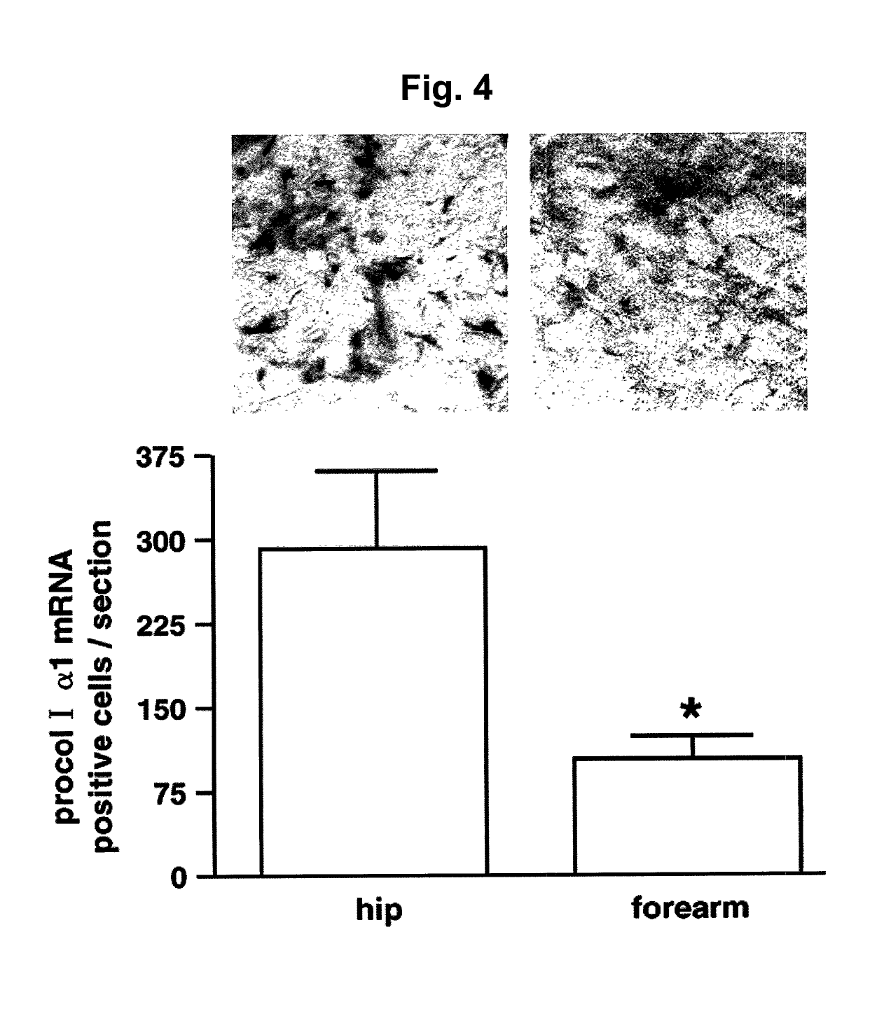

As described in our co-pending application Ser. No. 09/285,860, filed Apr. 2, 1999 (the disclosure of which is incorporated herein by reference), we again found a deficit in type I procollagen gene expression and type I procollagen protein expression in severely photodamaged skin versus sun-protected skin, as shown in FIG. 3. As described in that application, there is a double detriment to human skin from exposure to UV radiation: the induction of MMPs that degrade collagen, and the concurrent inhibition of collagen biosynthesis. With the present volunteers, we found that the number of cells expressing type I procollagen (α1) MRNA was reduced by approximately 65% in severely photodamaged forearm skin when compared with sun-protected skin from the hip; as shown in FIG. 4. Immunohistology results confirmed these findings: a significant reduction (about 57%) in the amount of type I procollagen in sun-exposed skin was found when it was compared with the amount of type I procollagen in sun-protected skin.

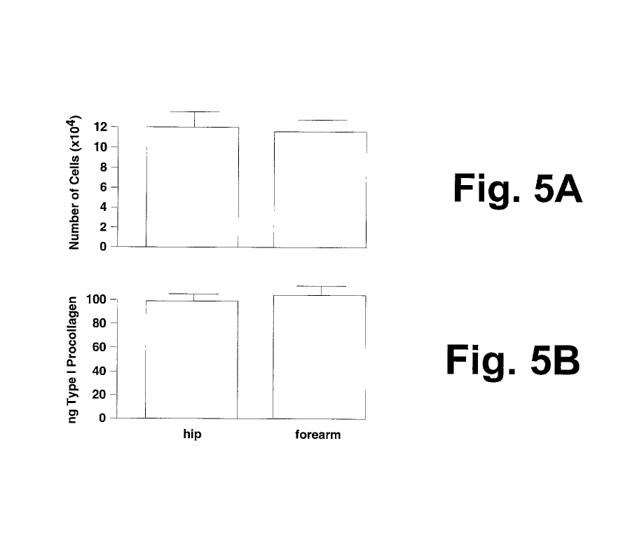

We next endeavored to determine further information about this reduction in collagen biosynthesis. A permanent incapacitation in fibroblasts of collagen synthetic activity in photoaged skin would explain these results, so we obtained a total of 36 fibroblast isolates from 108 fragments of photodamaged forearm skin (33%) and 43 isolates from 122 fragments of sun-protected hip skin (35%). Perhaps surprisingly, proliferation in vitro after two days resulted in a similar number of cells (FIG. 5A) and similar amounts of procollagen protein (FIG. 5B) from the isolates from both the forearm (sun-exposed) and the hip (sun-protected) skin. Accordingly, it appears that fibroblasts from sun-exposed human skin have not lost their ability to proliferate, nor to produce collagen.

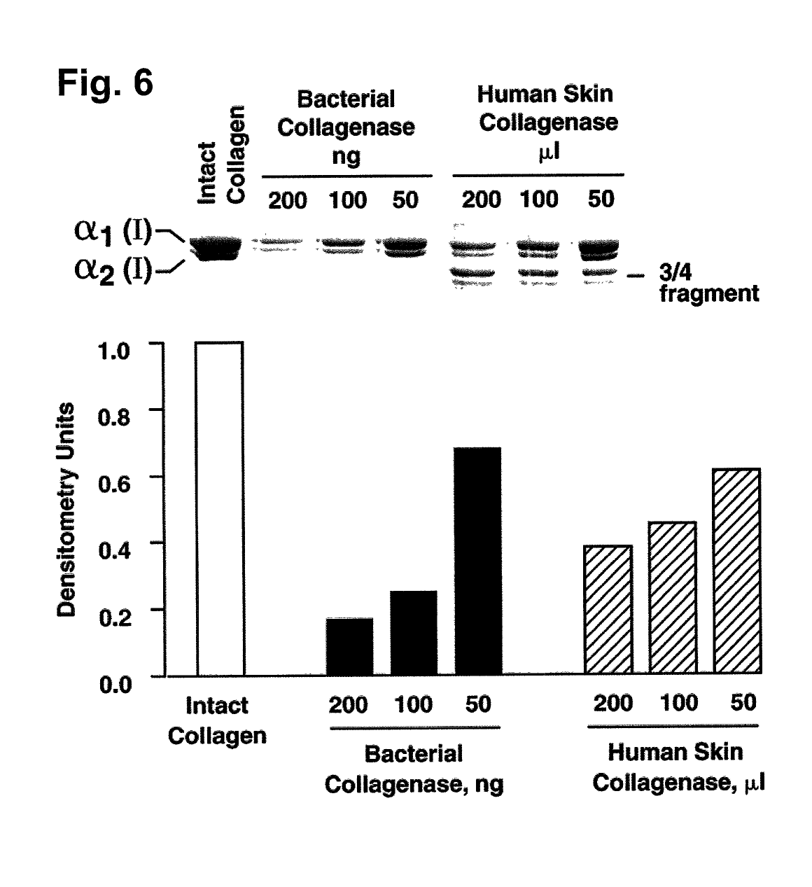

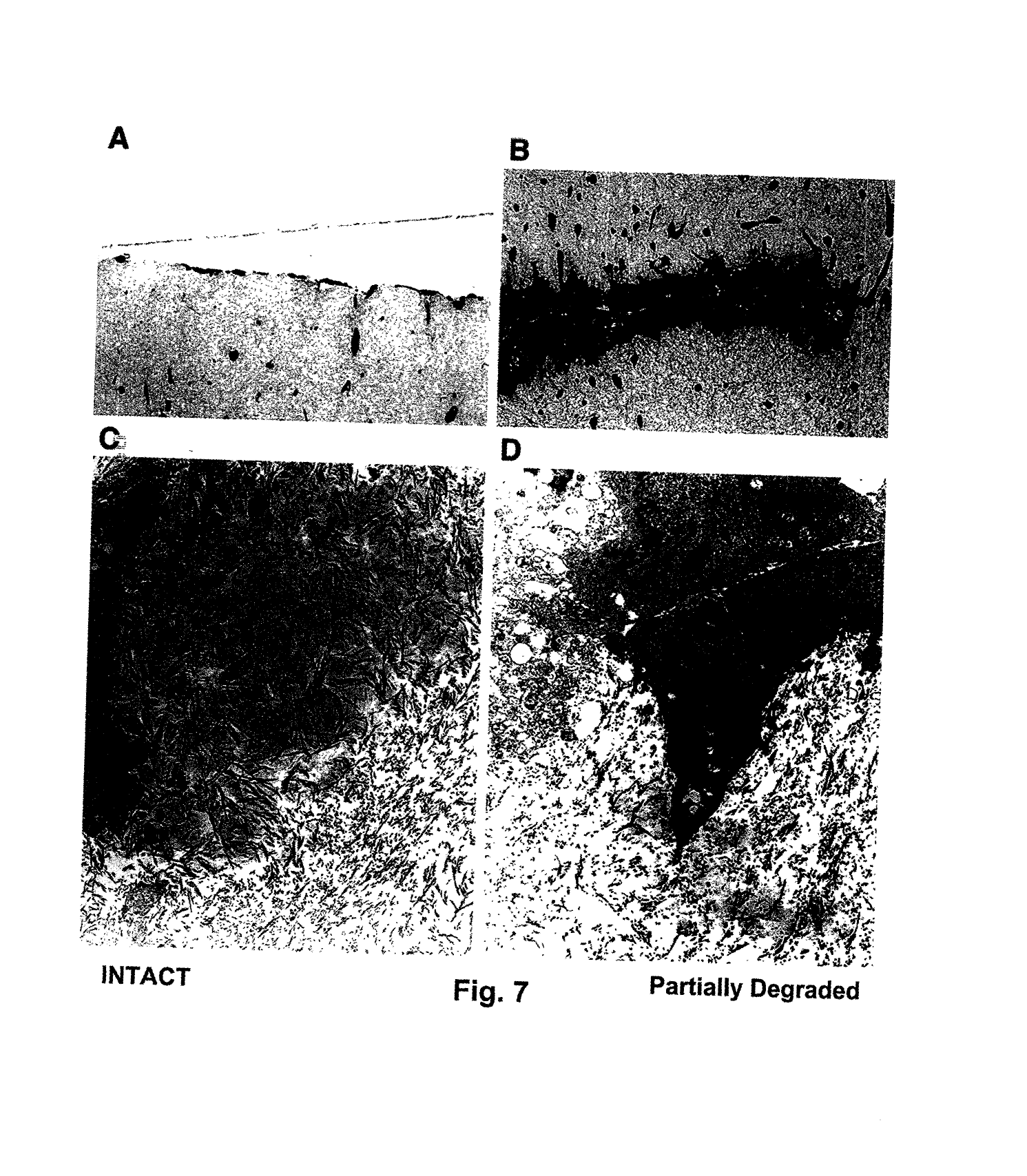

Accordingly, we endeavored to determine whether a factor in the fibroblasts' environment in vivo was inhibiting their ability to produce collagen. We prepared polymerized collagen gels (as described in the Methods and Materials section below) and treated these substrates with either bacterial collagenase or human skin collagenase from conditioned medium of basal cell carcinomas. We conducted experiments to determine the ability of the bacterial collagenase and human skin collagenase to degrade monomeric collagen, the results of which are shown in FIG. 6. Based on these results we standardized enzyme concentrations.

Collagen gels were separately treated with high concentrations of the enzymes, up to 200 ng of the bacterial collagenase per gel and 200 μl of the tumor culture fluid per gel. The gels remained polymerized and appeared indistinguishable from untreated gels. Dermal fibroblasts (8×10⁴ cells) were plated onto both untreated and treated/partially degraded collagen gels. During the subsequent one to two days, cells spread over the surface of the untreated gel and some migrated into the gel. On the treated gels, the cells also initially spread over the surface and migrated into the gel, and then cell-cell aggregation occurred and, as it did, the collagen contracted around the aggregated cells. These results are shown in FIGS. 7A and 7B. In FIG. 7A, a cross-section through a plated, untreated gel, the fibroblasts can be seen on the upper surface and other are relatively uniformly dispered in the gel. In contrast, in FIG. 7B, a cross section, the cell-cell aggregation can be seen. FIG. 7C shows a view of fibroblasts with their numerous processes under electron microscope on untreated gel; the fibroblasts were in close and frequent contact with the collagen fibers. As seen in FIG. 7D, fibroblasts surrounded by the collagen degradation debris showed decreased process formation, and there were few contacts between the cells and intact collagen fibers, again in contrast to untreated gel. In essence, the fibroblasts on the treated (degraded) collagen had become separated from intact collagen by the degradation debris.

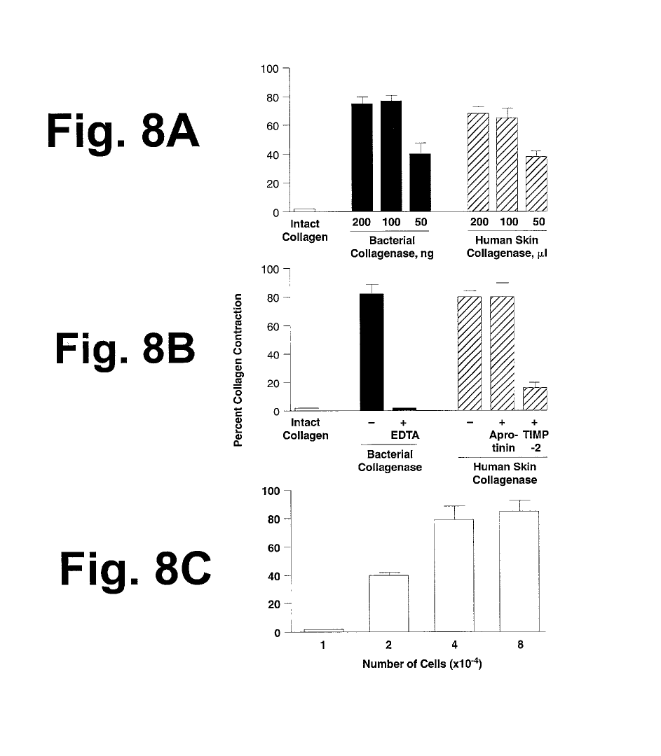

FIGS. 8A-8C demonstrate the relationship between collagen degradation and fibroblast activity. All three figures use collagen contraction (i.e., shortening of the collagen chain, collagen degradation) as an endpoint. FIG. 8A shows the dose-dependent relationship of the collagenases to the amount of collagen contraction achieved. FIG. 8B presents evidence that the metalloproteinases applied to the collagen gels were the cause of the collagen contraction. When the gel was exposed to bacterial collagenase plus 10 mM ethylene diamine tetraacetic acid (EDTA), and the EDTA was then neutralized with a source of calcium ion (Ca²⁺), there was no collagen contraction. Similarly, when gels were treated with the human skin collagenase in the presence of either 10 μg of aprotinin or 10 g human recombinant tissue inhibitor of metalloproteinase-2 (TIMP-2) collagen contraction was inhibited by TIMP-2 but not by aprotinin. FIG. 8C shows that collagen contraction was dependent on fibroblast activity: essentially full contraction occurred with 4-8×10⁴ cells, partial contraction occurred with as few as 2×10⁴ cells, and contraction was not observed with about 1×10⁴ cells.

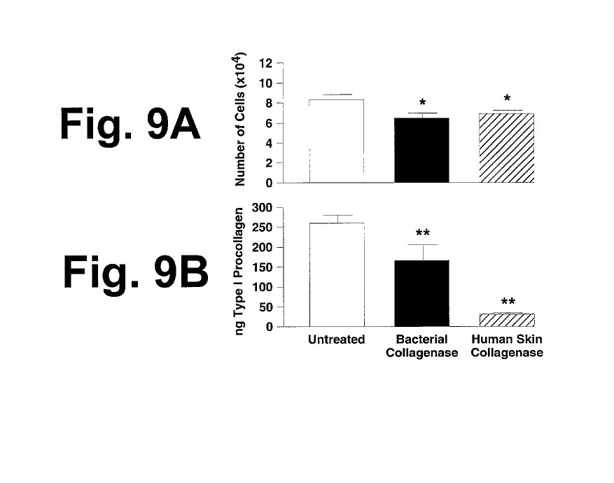

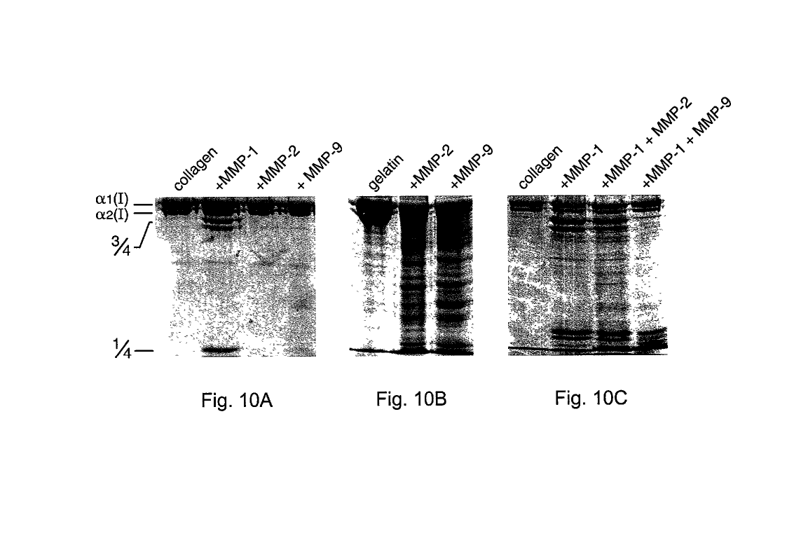

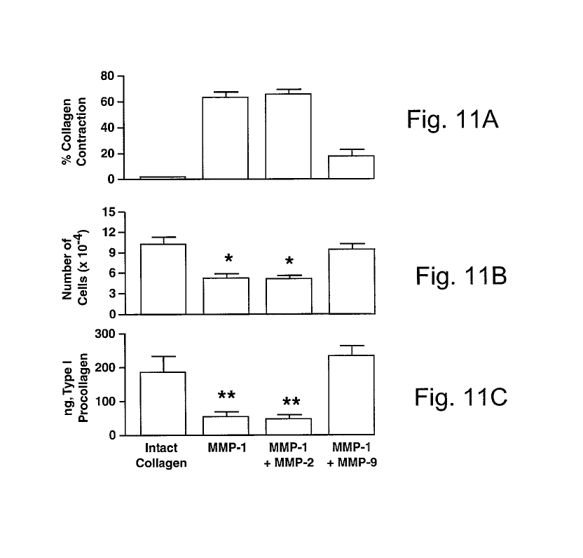

Cell growth and type I procollagen production by fibroblasts on intact collagen gels and gels partially degraded by either bacterial collagenase or human collagenase enzyme were assessed. For these studies, four different adult isolates (two from forearm and two from hip) and five different isolates of neonatal (foreskin) fibroblasts were used. Both cell growth (shown in FIG. 9A) and the amount of type I procollagen produced (shown in FIG. 9B) were lower on partially degraded collagen than on intact collagen, regardless whether the collagen was degraded by the bacterial collagenase or the human skin collagenase. Reduced cell growth and type I procollagen elaboration were observed with all of the neonatal and adult dermal fibroblast isolates tested.

FIGS. 10A-10C and FIGS. 11A-11C provide further evidence of the effects of different MMPs on collagen contraction. The gels of FIGS. 10A-10C were resolved by SDS-PAGE. Lane 1 of FIG. 10A is intact collagen and two bands, the α1(I) and α2(I) bands, are visible. Lane 2 of FIG. 10A shows the ¾ and ¼ fragments after digestion of collagen by MMP-1. Lanes 3 and 4 of FIG. 10A show that MMP-2 and MMP-9 did not degrade the collagen as did MMP-1. As noted above, MMP-2 and MMP-9 are gelatinases, not collagenases. Gelatin is made by heating collagen to 60° C. for five minutes. This processing unravels the collagen tri-helix and exposes numerous sites for enzymatic degradation. Lane 1 of FIG. 10B shows the resulting gelatin. Lanes 2 and 3 of FIG. 10B show the degradation products when gelatin is exposed, respectively, to MMP-2 and to MMP-9. Each of these gelatinases cleaves the original gelatin and also cleaves the fragments resulting from its cleavage of the gelatin, and so on.

FIG. 10C, although based on actual in vitro results, is a simulation of the in vivo effects of the presence of both a collagenase and a gelatinase. Lane 1 of FIG. 10C is the control collagen, and Lane 2 shows the degradation products when MMP-1 is present. Lane 3 shows the degradation products of collagen when both MMP-1 and MMP-2 are present. Comparing Lanes 2 and 3 of FIG. 10C with Lane 2 of FIG. 10A, it can be seen that the degradation products, the ¾ and ¼ fragments, are present in all; these are the degradation products of subjecting collagen to MMP-1. Lane 4 of FIG. 10C shows the degradation products of collagen when both MMP-1 and MMP-9 are present; namely, the ¾ and ¼ fragments produced by MMP-1 disappear. The MMP-9 activity is dose- and time-dependent; it may be present concommitant or subsequent to the treatment of the collagen with MMP-1. We are unsure why the MMP-2 used with the MMP-1 did not provide the same results as the combination of MMP-9 with MMP-1, and it may be that a higher concentration and/or longer incubation time would have provided those results.

The data from FIGS. 10A-10C are more relevant when viewed in combination with the results shown in FIGS. 11A-11C. Using the same procedures as used in the above experiments, we determined the responses of fibroblasts incubated on intact collagen, collagen treated with MMP-1, treated with a combination of MMP-1 and MMP-2, and treated with a combination of MMP-1 and MMP-9. FIGS. 11A through 11C show, respectively, the ability of the fibroblasts to contract the collagen (11A), the proliferation of the fibroblasts (11B), and their production of procollagen (11C). As seen in these three figures, the presence of MMP-1 alone decreases the proliferation and ability of the fibroblasts to make procollagen, and increases collagen contraction. However, the presence of MMP-9 in combination with MMP-1 negates these effects by allowing fibroblast proliferation and procollagen production and preventing collagen contraction.

Materials and Methods

Electron microscopy. Skin biopsies from forearm and hip skin were fixed overnight in 4% electron microscopic-grade glutaraldehyde in 0.1M cocodylate buffer at pH 7.4. After post-fixation with 2% osmium tetroxide buffered in 0.1M cocodylate buffer, sections were dehydrated with graded alcohol to 2×100% alcohol and 2× propylene oxide. The samples were embedded in pure epon resin. One micron tissue sections were cut, stained with Toluidine blue and examined at the light microscopic level. Ultrathin sections were cut from areas of interest, stained with lead citrate and uranyl acetate and observed in a Phillips 400 transmission electron microscope.

Assessment of collagen degradation in human skin. Skin biopsies from forearm and hip skin were homogenized in Tris buffer (20 mM, pH 7.3) and centrifuged. The pellet, containing the collagenous extracellular matrix, was resuspended in 150 μl of Tris buffer containing 75 μg of α-chymotrypsin, and incubated for 8 hours at 37° C. The pellet from homogenized skin biopsies incubated in buffer alone served as control. At the end of the incubation period, the reaction tubes were centrifuged at 10,000×g for 10 minutes. Supernatants were collected and assayed for hydroxyproline using automated amino acid analysis. Unlike intact fibrillar collagen, partially degraded collagen can be further broken down and the hydrolysis products liberated from tissue by α-chymotrypsin. The amount of released collagen-hydrolysis product can be determined by measurement of hydroxyproline, which is a modified amino acid present in collagen but rarely found in other proteins.

Assessment of type I procollagen synthesis in human skin in vivo. Assays for type I procollagen mRNA and protein were used to identify and quantify collagen-elaborating cells in skin samples. Type I procollagen (α1) gene expression was assessed by in situ hybridization. Fresh skin samples were immersed in OCT and frozen in liquid nitrogen. Frozen sections (6 μm) were hybridized with digoxigenin-labeled antisense and sense type I procollagen α1 cRNA probes. Cells expressing type I procollagen (α1) mRNA were quantified by counting under light microscopy. Type I procollagen protein was assessed by immunohistology. Frozen sections (6 μm) were stained with either one of two mouse monoclonal antibodies (SP1.D8, and M38) to human type I procollagen (α1 chain) and an immunoperoxidase-conjugated secondary antibody. The SP1.D8 antibody was developed by Dr. Heinz Furthmayr and obtained from the Developmental Studies Hybridoma Bank under the auspices of the NICHD and maintained by the Department of Biological Sciences, University of Iowa, Iowa City, Iowa 52242. The M38 antibody was obtained from Takara Biomedicals; Shiga, Japan. Stained sections were examined by light micro-scopy. The amount of cellular staining was assessed visually and scored as 0, 0.25, 0.5, 0.75 or 1.

Quantitative fibroblast outgrowth assay. Skin samples were cut into small fragments (12-15 fragments per 4 mm biopsy) and each fragment placed in a separate well of a 96-well plate. Tissue fragments were incubated for up to one month in Dulbecco's modified minimal essential medium of Eagle with non-essential amino acids and 10% fetal bovine serum (DMEM-FBS) at 37° C. in a humidified atmosphere containing 5% CO₂. The number of tissue fragments which yielded fibroblasts was determined at the end of the incubation period and expressed as a percentage of the total number of tissue fragments incubated. Cells were defined as fibroblasts on the basis of spindle-shaped morphology, reactivity with antibodies to vimentin, and a lack of reactivity with antibodies to keratin. Fibroblasts isolated in this manner were used without subculture or passaged 1-2 times before use.

Assessment of type I procollagen synthesis and fibroblast proliferation in vitro. Fibroblasts cultured from photodamaged forearm and sun-protected hip skin were plated in DMEM-FBS at 8×10⁴ cells per well in a 24-well culture plate. After allowing the cells to attach and spread, cells were washed twice in MCDB-153 basal medium (Clonetics Inc., Walkersville, Md.), supplemented with 1.4 mM Ca²⁺ (final concentration) and incubated for two days at 37° C. and 5% CO₂. At the end of the two day incubation period, cells were washed twice in Ca²⁺-supplemented MCDB-153 and incubated for an additional one hour at 37° C. and 5% CO₂. The one-hour culture fluid was collected and analyzed for type I procollagen protein by enzyme-linked immunoassay (Takara Biomedicals). Preliminary studies showed that the rate of accumulation of immunoreactive type I procollagen in medium conditioned by 8×10⁴ dermal fibroblasts was linear through at least two hours. After collection of the culture medium, cells were harvested by brief exposure to trypsin/EDTA and counted with the aid of a particle counter.

Preparation of polymerized collagen gels. Rat tail collagen (4.7 mg/ml in 1 N HCl) (Collaborative Biomedical Products, Bedford, Mass.) was diluted to 1 mg/ml with Ca²⁺ -supplemented MCDB-153. The solution was made isotonic by addition of an appropriate amount of a 10× concentrated solution of Hanks' Balanced Salt Solution, and the pH brought to 7.2. The collagen solution was added to wells of a 24-well plate (0.5 ml/well) and incubated for 2 hours at 37° C. During this period, the collagen formed a polymerized gel.

Collagen-degrading enzyme preparations. A collagenolytic enzyme preparation from Clostridium histolyticum (Collagenase type I; Worthington Biochemical Corp, Freehold, N.J.) was used to produce fragmentation of the collagen. This enzyme preparation contains collagenolytic activities at 105 and 55 kD, and the presence of these activities was confirmed by reactivity with gelatin and monomeric collagen, but not with β-casein in zymography. Reactivity was lost when 10 mM EDTA was included in the overnight incubation buffer. The bacterial enzyme preparation cleaves intact collagen at numerous sites to produce low molecular weight fragments. Collagenolytic activity was quantified by exposing 1 mg of rat tail (monomeric) collagen to varying concentrations of enzyme preparation for 5 hours at 37° C. Intact collagen exposed to buffer alone served as control. At the end of the incubation period, the control collagen and enzyme-treated collagen were resolved on SDS-PAGE and stained with Coomassie brilliant blue. Laser densitometry was used to quantify α1(I) and α2(I) bands in the intact and digested preparations. When 10 mM EDTA was included in the reaction mixture, no detectable collagen breakdown occurred. Human basal cell carcinoma tissue was used as a source of collagen-degrading enzymes from human skin. Fresh tumor specimens obtained at surgery were cut into 2-mm pieces, and 6-8 tissue pieces incubated for 72 hours in 0.5 ml of Ca²⁺-supplemented MCDB-153. Incubation was at 37° C. and 5% CO₂. At the end of the incubation period, the culture fluid was obtained and used as the enzyme source. The conditioned medium from basal cell tumors contains large amounts of active MMP-1 as well as small amounts of MMP-8 (neutrophil collagenase) and MMP-13 (collagenase-3). Active forms of gelatinolytic enzymes (e.g., MMP-2 and MMP-9) are also present. Zymography with gelatin, collagen and β-casein was used in the present study to confirm the presence of these activities, and collagen-degrading activity was quantified using digestion of monomeric collagen followed by SDS-PAGE resolution as described above. As with the bacterial enzyme preparation, inclusion of 10 mM EDTA in the incubation buffer suppressed zymographic activities, and inclusion of 10 mM EDTA in the reaction mixture suppressed collagen-degradation.

Polymerized collagen gels were treated for 5 hours at 37° C. with varying amounts of either the bacterial enzyme or human skin enzyme preparation. At the end of the incubation period, the collagenase solutions were decanted. The polymerized collagen gels were briefly exposed in sequence to 10 mM EDTA and 14 mM Ca²⁺, and then rinsed exhaustively with Ca²⁺-supplemented MCDB-153.

Assessment of collagen contraction and type I procollagen sysnthesis by fibroblasts on polymerized collagen gels. Four isolates of adult fibroblasts (two from forearm and two from hip) and five isolates of neonatal foreskin fibroblasts at passage 1-2 were added to the collagen gels at a final concentration of 1-8×10⁴ cells per culture. For this, Ca²⁺-containing MCDB-153 medium was further supplemented with 0.1 ng/ml epidermal growth factor, 0.5 μg/ml insulin and 2% pituitary extract. Cells were incubated for 4 days, with fresh culture medium provided on day 2. Contraction of the collagen gels occurred over a 2-day period. The diameter of the collagen gel was measured at day-2 using a microscope with a calibrated grid in the eyepiece. Collagen contraction in this assay depends on fibroblasts binding to the collagen fibers and pulling the fibers as the cells, themselves, undergo actinand myosin sliding filament-mediated contraction.

At the end of the incubation period (day-4), the culture fluid was removed, and the collagen gels rinsed two times with Ca²⁺-supplemented MCDB-153 (without the added growth factors). Fresh culture medium (Ca²⁺-supplemented MCDB-153 without growth factors) was added to the wells and incubated for a further one hour. The one-hour culture fluid was collected and assayed for type I procollagen by ELISA as described above. The cells were then released from the collagen gels by sequential treatment with a high concentration of the bacterial collagenase preparation (100 μg for 2 hours) and trypsin (0.5% for 15 minutes) and counted.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A topical composition for protecting human skin from collagen degradation, comprising a compound selective for the inhibition of at least one of MMP-1, MMP-8, and MMP-13 with respect to MMP-9 and optionally MMP-2, and a dermatologically suitable carrier therefor.

2. The composition of claim 1, further comprising a retinoid.

3. The composition of claim 1, further comprising at least one of a UVA blocker and a UVB blocker.

4. The composition of claim 3, comprising both a UVA blocker and a UVB blocker.

5. The composition of claim 4, further comprising a retinoid.

6. The composition of claim 1, further comprising an antioxidant.

7. The composition of claim 6, further comprising both a UVA blocker and a UVB blocker.

8. A method for restoring collagen and fibroblast proliferation in chronologically aged skin, comprising topically apply a compound selective for the inhibition of at least one of MMP-1, MMP-8, and MMP-13 with respect to MMP-9 and optionally MMP-2, and a dermatologically suitable carrier therefor.

9. The method of claim 8, further comprising topically applying to the skin a retinoid.

10. The method of claim 8, wherein the application is made daily.

11. The method of claim 9, wherein the application is made daily.

12. A method for preventing collagen degradation and improving procollagen biosynthesis inhibition in human skin due to exposure of said skin to UV radiation, comprising topically applying a compound selective for the inhibition of at least one of MMP-1, MMP-8, and MMP-13 with respect to MMP-9 and optionally MMP-2, and a dermatologically suitable carrier therefor, between 24 hours prior to said exposure to twelve hours after said exposure.

13. The method of claim 12, wherein the application is prior to UV exposure, and further comprising apply a combination of UVA and UVB blockers.

14. The method of claim 12, wherein the application is prior to UV exposure, and further comprising applying an EGF-R protein tyrosine kinase inhibitor.

15. A method for treating acne, comprising topically applying a compound selective for the inhibition of at least one of MMP-1, MMP-8, and MMP-13 with respect to MMP-9 and optionally MMP-2, and a dermatologically suitable carrier therefor.
